(19) United States
(12) Patent Application Publication
(10) Pub. No.: US 2025/0281076 A1
(43) Pub. Date: Sep. 11, 2025

(54) Systems and Methods for Integrated Spinal Cord Stimulation and Glucose Monitoring

(71) Applicant: Glucotrack, Inc., Rutherford, NJ (US)

(72) Inventors: Paul V. Goode, Temple, TX (US); Mark Allan Tapsak, Front Royal, VA (US); James Patrick Thrower, Bethesda, MD (US)

(21) Appl. No.: 19/076,714

(22) Filed: Mar. 11, 2025

Related U.S. Application Data
(60) Provisional application No. 63/563,880, filed on Mar. 11, 2024.

(51) Int. Cl. A61B 5/145; A61B 5/00; A61B 5/1455; A61B 5/1473; A61B 5/1486; A61N 1/05; A61N 1/36; A61N 1/372; A61N 1/378

(52) U.S. Cl. CPC A61B 5/14532; A61B 5/1455; A61B 5/14735; A61B 5/14865; A61B 5/686; A61N 1/0551; A61N 1/36062; A61N 1/36071; A61N 1/36125; A61N 1/37211; A61N 1/378; A61B 2560/0214; A61B 2560/0468

(57) ABSTRACT

A system for continuous glucose measurement and spinal cord stimulation provides at least two implantable non-stimulation electrodes configured to continuously monitor at least one physiological parameter on a lead that also includes a plurality of implantable stimulation electrodes. The electrodes are electrically connected to an implantable pulse generator that communicates stimulation instructions and glucose measurement data.

### CROSS-REFERENCE

The present application relies on, for priority, U.S. Patent Provisional Application No. 63/563,880, titled “Systems and Methods for Integrated Spinal Cord Stimulation and Glucose Monitoring” and filed on Mar. 11, 2024, which is herein incorporated by reference in its entirety.

### FIELD

The present specification relates to the field of monitoring physiological parameters in a patient. Specifically, the present specification relates to devices and treatment protocols for continuously monitoring analytes, such as a glucose level of a patient, by positioning the analyte sensor within, or proximate to, the spinal column of a patient.

### BACKGROUND

Research indicates that approximately 20% of people with diabetes are likely to develop painful diabetic neuropathy (PDN) with paresthesia, burning, and/or shooting pain. Patient compliance and adherence to taking commonly prescribed PDN medications are poor due to inadequate pain relief or intolerable side effects. Recently, spinal cord stimulation (SCS) systems have been designed to treat PDN. Spinal cord stimulators (SCS) use programmable pulse generation systems that deliver electrical stimulation through electrodes or arrays of electrodes that are implanted in the epidural space near the spine of a patient. SCS systems include an implantable, programmable pulse generator that generates electrical pulses transmitted through leads to epidurally implanted electrodes. Pulse amplitude, polarity, and frequency, among other parameters, are governed by programming signals. The programming signals may be communicated by an external computing device that is in communication with the implantable programmable pulse generator.

In addition to addressing the neuropathy of diabetes sufferers, there is a need to continuously measure glucose levels for diabetic patients, and specifically patients diagnosed with PDN. There are many different types of glucose sensors used for continuous monitoring of glucose levels. Implantable glucose sensors may be subcutaneous or intravascular. Percutaneous glucose sensors may also be subcutaneous or intravascular. Glucose monitoring may be effectuated using a variety of different sensing modalities, including enzymatic sensing, fluorescence sensing, and optical sensing. Continuous glucose monitors typically include a glucose electronics assembly and a glucose lead assembly in electrical communication with the glucose electronics assembly.

What is needed is a system that effectively combines a SCS system with a continuous glucose monitoring system which can reduce the burden of using two separate devices and can provide a more efficiently deployed system to concurrently monitor the glucose levels of, and treat the pain experienced by, PDN patients.

### SUMMARY

The following embodiments and aspects thereof are described and illustrated in conjunction with systems, tools and methods, which are meant to be exemplary and illustrative, and not limiting in scope. The present application discloses numerous embodiments.

In some embodiments, the present specification discloses a spinal cord stimulation system, comprising: a plurality of implantable stimulation electrodes positioned on a substrate; at least two implantable non-stimulation electrodes configured to continuously monitor at least one physiological parameter, wherein the at least two implantable non-stimulation electrodes are positioned on said substrate; an implantable pulse generator; at least one implantable lead electrically connecting the plurality of implantable stimulation electrodes and the at least two implantable non-stimulation electrodes to the implantable pulse generator.

Optionally, the at least two implantable non-stimulation electrodes comprise at least a working electrode and at least a reference electrode. Optionally, the at least two implantable non-stimulation electrodes further comprise a counter electrode.

Optionally, the reference electrode comprises at least one of a silver substrate or a platinum-iridium substrate.

Optionally, a first surface area of the counter electrode is greater than a second surface area of the working electrode.

Optionally, the working electrode comprises a polymer-based enzyme coating. Optionally, the working electrode further comprises a polymer-based coating over the polymer-based enzyme coating.

Optionally, the system comprises two sets of the at least two implantable non-stimulation electrodes, wherein the two sets are configured for differential measurement of the at least one physiological parameter.

Optionally, the at least one physiological parameter comprises glucose levels.

Optionally, the at least two implantable non-stimulation electrodes are configured to continuously monitor the least one physiological parameter within an epidural space.

Optionally, the implantable pulse generator comprises a lead assembly, wherein the lead assembly comprises electrical contacts for electrical communication with the plurality of implantable stimulation electrodes and with the at least two implantable non-stimulation electrodes. Optionally, the implantable pulse generator comprises at least one of a potentiostat, an analog to digital converter, a power source, a digital communication circuit, or a microcontroller. Still optionally, the implantable pulse generator comprises a power source that is at least one of rechargeable or non-rechargeable. Still optionally, the implantable pulse generator comprises a programmable electronic circuitry configured to communicate with at least one computing device.

Optionally, the at least two implantable non-stimulation electrodes form a sensor for continuously monitoring glucose levels in a patient, wherein the sensor is at least one of an enzymatic sensor or a non-enzymatic sensor. Optionally, the enzymatic sensor comprises at least one of a wired enzymatic sensor, an engineered enzymatic sensor, an H2O2 based enzymatic sensor, or an O2 differential based enzymatic sensor. Optionally, the non-enzymatic sensor comprises at least one of a photodetector or a glucose binding molecule.

Optionally, the at least two implantable non-stimulation electrodes comprise one or more bioresorbable membranes.

In some embodiments, the present specification describes a continuous physiological parameter monitoring device integrated with a spinal cord stimulation system, comprising: a plurality of implantable stimulation electrodes positioned on a substrate; at least two implantable non-stimulation electrodes configured to continuously monitor at glucose levels positioned on said substrate; an implantable pulse generator; at least one implantable lead electrically connecting the plurality of implantable stimulation electrodes and the at least two implantable non-stimulation electrodes to the implantable pulse generator.

In some other embodiments, the present specification discloses a spinal cord stimulation system, comprising: a plurality of implantable stimulation electrodes positioned on a substrate; at least one implantable non-stimulation electrode configured to continuously monitor at least one physiological parameter, wherein the at least one implantable non-stimulation electrodes are positioned on said substrate; an implantable pulse generator; at least one implantable lead electrically connecting the plurality of implantable stimulation electrodes and the at least one implantable non-stimulation electrode to the implantable pulse generator.

Optionally, the spinal cord stimulation system of claim 21 wherein the at least one implantable non-stimulation electrodes comprises a working electrode.

Optionally, the spinal cord stimulation system further comprises at least two non-stimulation electrodes that are not implanted. Optionally, the at least two non-stimulation electrodes that are not implanted further comprise a reference electrode and a counter electrode.

The aforementioned and other embodiments of the present specification shall be described in greater depth in the drawings and detailed description provided below.

### BRIEF DESCRIPTION OF THE DRAWINGS

The accompanying drawings illustrate various embodiments of systems, methods, and embodiments of various other aspects of the disclosure. Any person with ordinary skills in the art will appreciate that the illustrated element boundaries (e.g. boxes, groups of boxes, or other shapes) in the figures represent one example of the boundaries. It may be that in some examples one element may be designed as multiple elements or that multiple elements may be designed as one element. In some examples, an element shown as an internal component of one element may be implemented as an external component in another and vice versa. Furthermore, elements may not be drawn to scale. Non-limiting and non-exhaustive descriptions are described with reference to the following drawings. The components in the figures are not necessarily to scale, emphasis instead being placed upon illustrating principles.

FIG. 1A illustrates a spinal cord stimulation (SCS) system using a paddle lead;

FIG. 1B illustrates a SCS system using an isodiametric lead;

FIG. 2 illustrates an embodiment of a lead configuration including a SCS electrode array for stimulation and a glucose electronic assembly for sensing glucose levels, in accordance with an exemplary embodiment of the present specification;

FIG. 3 illustrates an embodiment of a lead configuration including a SCS electrode array for stimulation and a glucose electronic assembly for sensing glucose levels, in accordance with an exemplary embodiment of the present specification;

FIG. 4 illustrates another embodiment of a lead configuration including a SCS electrode array for stimulation and a glucose electronic assembly for sensing glucose levels, in accordance with an exemplary embodiment of the present specification;

FIG. 5 illustrates another embodiment of a lead configuration including a SCS electrode array for stimulation and a glucose electronic assembly for sensing glucose levels, in accordance with an exemplary embodiment of the present specification;

FIG. 6 illustrates another embodiment of a lead configuration including a SCS electrode array for simulation and a glucose electronic assembly for sensing glucose levels, in accordance with an exemplary embodiment of the present specification; and

FIG. 7 illustrates a portion of a lead configuration that is positioned within the epidural space, further showing a cross-section of the lead, in accordance with an exemplary embodiment of the present specification.

### DETAILED DESCRIPTION

The present specification is directed towards methods and systems to integrate continuous glucose measurement (CGM) with spinal cord stimulation (SCS). In embodiments, the patient is monitored for an analyte other than glucose. Systems of the present specification are configured to enable a patient to receive therapy or treatment for conditions while receiving data from analyte monitoring. In embodiments, an analyte sensing membrane is positioned on an SCS lead such that the analyte is measured and the data is communicated to a computing device which is in communication with the SCS. In embodiments, the systems of the present specification provide for placement of CGM electrodes and sensing membrane chemistry within an SCS device. Thus, systems of the present specification are configured to enable a patient to receive therapy or treatment for conditions such as Painful Diabetic Neuropathy (PDN) while receiving data from continuous glucose monitoring (CGM). In embodiments, the computing device includes a display and may be operated by either or both a physician/health care provider and the patient. The present specification discloses methods and systems that can be used with any patient with diabetes or pre-diabetes that are using SCS for any reason including and not limited to pain relief, urinary incontinence, fecal incontinence, and movement.

The present specification is directed towards multiple embodiments. The following disclosure is provided in order to enable a person having ordinary skill in the art to practice the invention. Language used in this specification should not be interpreted as a general disavowal of any one specific embodiment or used to limit the claims beyond the meaning of the terms used therein. The general principles defined herein may be applied to other embodiments and applications without departing from the spirit and scope of the invention. Also, the terminology and phraseology used is for the purpose of describing exemplary embodiments and should not be considered limiting. Thus, the present invention is to be accorded the widest scope encompassing numerous alternatives, modifications and equivalents consistent with the principles and features disclosed. For purpose of clarity, details relating to technical material that is known in the technical fields related to the invention have not been described in detail so as not to unnecessarily obscure the present invention.

In the description and claims of the application, each of the words “comprise”, “include”, “have”, “contain”, and forms thereof, are not necessarily limited to members in a list with which the words may be associated. Thus, they are intended to be equivalent in meaning and be open-ended in that an item or items following any one of these words is not meant to be an exhaustive listing of such item or items, or meant to be limited to only the listed item or items. It should be noted herein that any feature or component described in association with a specific embodiment may be used and implemented with any other embodiment unless clearly indicated otherwise.

It must also be noted that as used herein and in the appended claims, the singular forms “a,” “an,” and “the” include plural references unless the context dictates otherwise. Although any systems and methods similar or equivalent to those described herein can be used in the practice or testing of embodiments of the present disclosure, the preferred, systems and methods are now described.

It should be noted that each component, processor, patient monitor, and/or device described herein is configured to perform the functions and communications described herein. In embodiments, each patient monitor, server, and/or system is capable of executing programmatic instructions. It should further be appreciated that each device and monitoring system have wireless and wired receivers and transmitters capable of sending and transmitting data, at least one processor capable of processing programmatic instructions, memory capable of storing programmatic instructions, and software comprised of a plurality of programmatic instructions for performing the processes described herein.

The present invention is now discussed in the context of components of an SCS device, in various embodiments.

### Lead

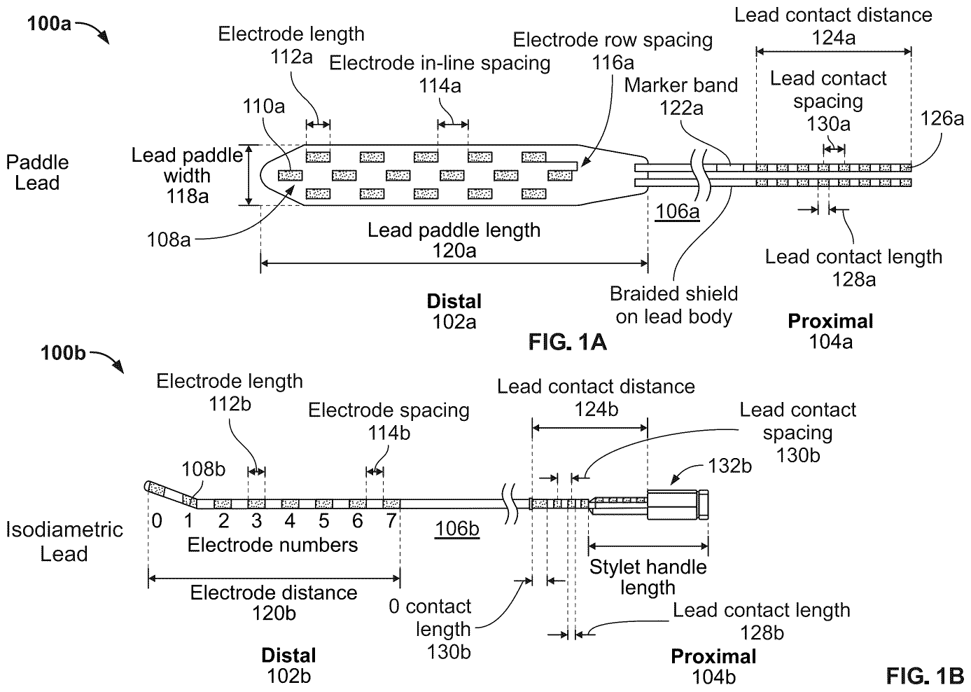

Leads are configured to carry electrical stimulation pulses that are generated by an implantable pulse generator (IPG) to which the leads are electrically connected through a lead contact system. A conventional SCS device comprises one or more leads that are inserted under the skin and subcutaneously fed to and inserted into the spinal canal. The leads contain one or more electrodes or array of electrodes. The leads may be of one of two types—a paddle lead and an isodiametric lead—both of which have a different construction and method of insertion. FIG. 1A illustrates a paddle lead system and FIG. 1B illustrates an isodiametric lead system.

Referring to FIG. 1A, paddle lead system 100a includes a distal section 102a and a proximal section 104a, which are connected to each other in the middle by connecting leads 106a that are covered with a braided shield. Distal section 102a comprises a paddle-shaped configuration with a width 118a and a length 120a that is greater than width 118a. Distal section 102a has a flat surface to accommodate multiple horizontal rows of electrodes (or electrode arrays) 108a. The illustrated example of paddle lead system 100a is shown with three rows of electrodes 108a. Each row has multiple electrodes 110a of equal lengths 112a aligned with an equal spacing 114a between them. In embodiments, the electrodes range from 0.5 mm to 8.0 mm in length. The rows 108a aligned along edges of distal section 102a are configured with electrodes 110a that are vertically aligned to each other, and the middle row has electrodes that are positioned at an offset distance in the vertical direction from the electrodes 110a of the other two rows. In embodiments, as an example of the offset distance, a first edge 150a of the most distal electrode in the middle row may be at a distance in a range from 0.1 mm to 5.0 mm from edges 150b of the most distal electrodes in the rows aligned along the edges.

Each row 108a is spaced equally from each other at a distance 116a. The beginning of proximal section 104a is marked with a marker band 122a positioned on a distal side of section 104a. Distance 124a, which ranges from 0.1 mm and 5.0 mm, over which lead contacts 126a are positioned, is proximal to marker band 122a. Over this distance 124a, lead contacts 126a, each of a length 128a are aligned in two parallel rows with a spacing 130a between each lead contact 126a within each row. In embodiments, the length 128a of each of the lead contacts 126a ranges from 0.5 mm to 8.0 mm. In embodiments, the spacing 130a between each lead contact 126a within each row ranges from 0.1 mm to 5.0 mm.

Referring to FIG. 1B, an isodiametric lead system 100b includes a distal section 102b and a proximal section 104b, which are connected to each other in the middle by a connecting lead 106b. Distal section 102b comprises an elongated tubular configuration with multiple electrodes (or electrode arrays) 108b of equal lengths 112b circumferentially configured around the tube at a space 114b from each other. In embodiments, length 112b ranges from 0.5 mm to 8.0 mm. In embodiments, space 114b is of equal length throughout the circumference. In embodiments, space 114b ranges from 0.1 mm to 5.0 mm. Electrodes 108b are positioned over a distance 120b, corresponding to a length of distal section 102b of isodiametric lead system 100b. The illustrated example of isodiametric lead system 100b is shown with eight electrodes 108b which are numbered from 0 to 7. Proximal section 104b has lead contacts 126b, each of length 128b, which are spaced equally at a distance 124b circumferentially around the tubular configuration of proximal section 104b. An exception is the lead contact length 130b corresponding to the most distal electrode (number 0), which is greater than other lead contact lengths 128b. A stylet handle 132b is positioned on a proximal side of proximal section 104b including lead contact distance 124b.

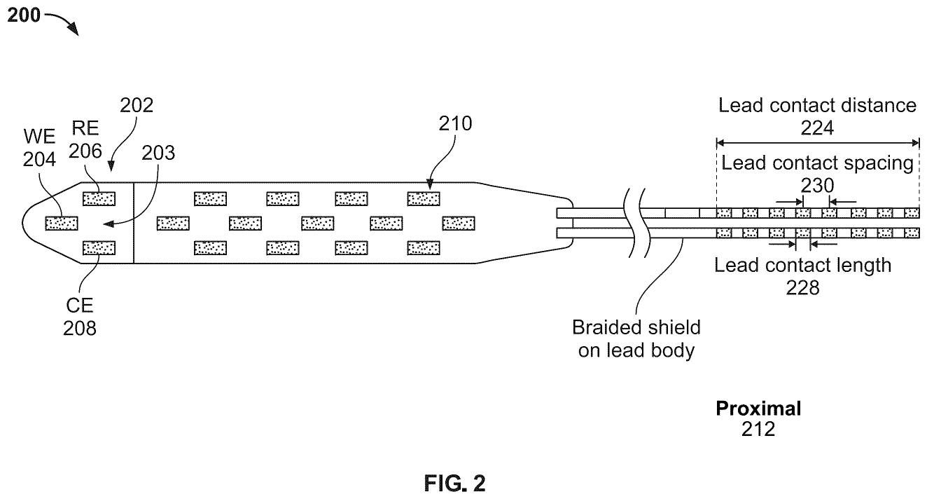

FIG. 2 illustrates an embodiment of a lead configuration including a SCS electrode array for stimulation and a glucose electronic assembly for sensing glucose levels, in accordance with an exemplary embodiment of the present specification. As shown in FIG. 2, paddle lead system 200 includes assigned electrodes 202 incorporated for analyte sensing within a SCS system, in accordance with an exemplary embodiment of the present specification. System 200 is a modified form of a paddle lead assembly of FIG. 1A. The changes made to an isodiametric lead assembly are similar to those described herein for the paddle lead assembly, with differences in the changes primarily due to differences in the constructions of the two types of lead assemblies. Analyte sensing elements are described hereinafter as specific to glucose monitoring, however, the embodiments of these elements can be used for sensing or monitoring any other analyte. In embodiments, assigned electrodes 202 comprise three dedicated electrodes that are preferably configured to not stimulate or sense electrical activity. Specifically, three assigned electrodes 202 can be referred to as follows: a working electrode (WE) 204, a reference electrode (RE) 206 and a counter-electrode (CE) 208. In embodiments, electrodes 202 are located proximate to each other. In some embodiments, electrodes 202 are located at the distal end of distal portion 203, within a tapered distal end of the paddle-shaped lead system. In order to increase the respective surface areas of each of electrodes 202, an abrasion process, such as grit blasting, laser-etching methods, chemical etching, or mechanical abrasion, such as grinding are used to increase the respective surface areas of each of electrodes 202. Increasing the surface area of the sensor substantially proportionally improves glucose sensing performance and membrane adhesion. In embodiments, as described in detail below, dip-coating is used for membrane deposition. In cases where dip-coating is employed, it is preferable for the dip coating to be applied near the distal portion 203 of the distal end of the paddle lead. In embodiments, the assigned electrodes 202 are positioned away from stimulating electrodes to mitigate noise on the sensor.

In embodiments, WE 204 has a polymer-based enzyme layer over it. The polymer-based enzyme layer is coated using one of the methods of dipping, spinning or casting. The polymer-based enzyme layer of WE 204 is coated while ensuring that all the other electrodes of a SCS lead device are masked and protected from the coating. Furthermore, WE 204 has a polymer-based layer over the polymer-based enzyme layer. The polymer-based layer is coated using one of the methods of dipping, spinning or casting. The additional polymer-based layer aids in limiting glucose diffusion and any other interfering species within WE 204. In some optional embodiments, one or both of RE 206 and CE 208 have similar layers as those of WE 204. In some optional embodiments, the RE 206 and CE 208 may be covered in part, in whole, or not covered at all by membranes.

In embodiments, RE 206 has either a silver (Ag) substrate or a platinum-iridium (PtIr) substrate. In the case of RE 206 comprising an Ag substrate, RE 206 is chloridized to form Ag/AgCl on its surface, either by electrodeposition or a chemical chloridization using, for example, sodium hypochlorite. Whereas, in the case of RE 206 comprising a PtIr substrate, an iridium oxide (IrOx) layer is formed on the surface of RE 206, either by electro-deposition or sputtering.

A surface area of CE 208 is preferably larger than a surface area of WE 204. Such a configuration can be achieved by shorting two or more electrodes 110a (see FIG. 1) that are either internal to the lead body proximally, distally, or in between, or with the IPG header electronics, or in between; or by manufacturing CE 208 larger than WE 204. In some embodiments, CE 208 has twice the surface area of WE 204. In one particular embodiment, namely the amperometric detection of H₂O₂, the working electrode receives 2 electrons for every H₂O₂ molecule that is reduced. The counter electrode must deliver those electrons back into the “system” by oxidizing a molecule such as oxygen. The reduction rate (and therefore, signal generation) at the working electrode can be influenced if the rate of oxidation reactions at the counter electrode become limiting. Therefore, to ensure that the counter electrode is not the rate limiting factor which would effectively limit measurements at the working electrode, the counter electrode is manufactured as larger than the working electrode.

In some embodiments, each or at least one of the electrodes have a surface area in a range of 5 mm² to 30 mm². In an embodiment, working electrode 204 has a surface area ranging from 8 mm² to 20 mm², is employed along with a reference electrode 206 and a counter electrode 208 with a surface area that is at least 1.5 times greater than the surface area of the working electrode 204. In another embodiment, working electrode 204 with a surface area ranging from 8 mm² to 20 mm² is employed along with a combined reference and counter electrode (not shown) with a surface area that is at least 1.5 times smaller than that of working electrode 204.

The lead system, in accordance with the present specification, is configured to enable a connection of electrodes 202 to an electrical contact corresponding to each of electrodes 202 in a proximal section 212 of the lead where the lead interfaces with an IPG, similar to the connection of other electrodes in an SCS lead to the contacts in the IPG. FIG. 2 illustrates the relative distance 224 over which the lead contacts are positioned in proximal section 212, the relative length 228 of each contact, and the relative spacing 230 between each adjacent contact in a paddle lead device. Each corresponding contact in proximal section 212 is configured to deliver signals sensed by electrodes 202 to the IPG when the lead is inserted into the spinal column.

In some embodiments, the device includes three electrodes 202. In embodiments, a three-electrode configuration lends to a greater accuracy and stability for long life. In some embodiments, two electrodes 202 are sufficient to sense glucose, is easier to manufacture, has reduced parts, however is less stable over long time periods (months to years). In an embodiment with a two electrodes configuration, a WE and a RE are used, where the size of the RE is at least twice the size of the WE. A two-electrode configuration requires a fewer number of electrodes and, therefore, connections. In some embodiments, either a two-electrode or three-electrode configuration is used for a differential approach to glucose monitoring, whereby the differential measurement is achieved by looking at two slightly different WE membranes. A differential measurement can result in reducing signal noise due to interferents and noise sources common to both electrode sets. This approach, however, requires twice as many electrodes. In other embodiments, the device includes four electrodes 202.

As described with reference to the figures, the sensors and components described in the present specification include key functionalities as described herein due to the unique combination of dimensions, thicknesses, flexibility, materials used, and positioning of certain elements. Therefore, the use and combination of these parameters should not be construed as mere design choices and, rather, should be accorded patentable weight. It should also be noted that the various components described herein may be used with any other component as described herein, in any combination or order, even if not described with respect to certain embodiments. Further, it should be noted that the various parameters described herein may apply without restriction to the embodiments in which they are described. Therefore, the components and parameters described throughout this specification are interchangeable and may be combined to achieve the objectives of the present invention and not limited to the specific embodiments.

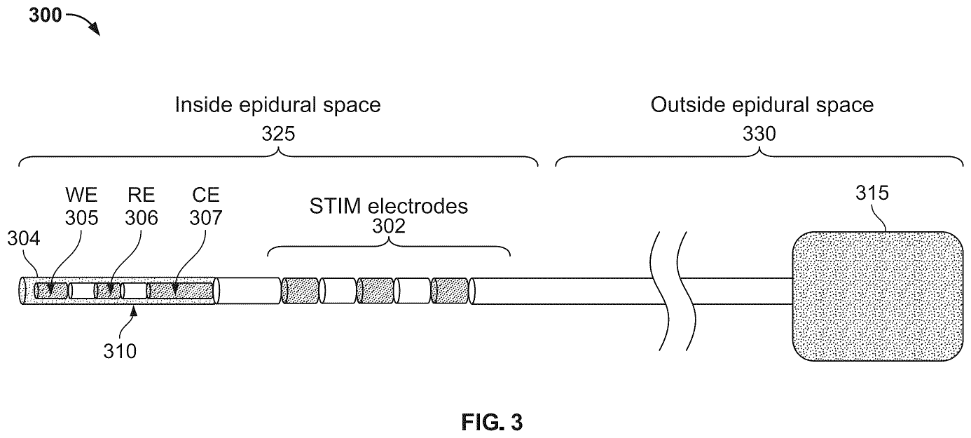

FIG. 3 illustrates an embodiment of a lead configuration 300 including a SCS electrode array 302 for stimulation and a glucose electronic assembly 304 for sensing glucose levels, in accordance with an exemplary embodiment of the present specification. In embodiments, the lead body comprising electrode array 302 and glucose electronic assembly 304 are in electrical communication with an electronics assembly 315. Further, the lead body comprising electrode array 302 and glucose electronic assembly 304 form a first portion 325 of lead configuration 300, which is positioned inside an epidural space; electronics assembly 315 is comprised within a second portion 330 of lead configuration 300 and is positioned outside the epidural space. In some other embodiments, and as described in United States Patent Publication No. 20230079720, assigned to the Applicant of the present invention and herein incorporated by reference in its entirety, electronics assembly 315 is configured to be positioned in the subcutaneous tissue and glucose electronics assembly 304 is configured to be positioned in a vessel of the patient.

In embodiments, a length of the lead body comprising electrode array 302 and glucose electronic assembly 304 is in a range of 80 mm to 260 mm, and in one embodiment the length is approximately 171 mm. In embodiments, the length of the lead body may extend beyond 171 mm. In embodiments, a length of the electronics assembly 315 is in a range of 30 mm to 80 mm, and in one embodiment the length is approximately 60 mm.

In embodiments, the length of lead body used herein is much shorter than a pacing lead and much longer than that used in a conventional subcutaneous sensor. In some embodiments, a length of lead extending from its distal tip to its proximal end where the lead body connects with the electronics assembly may be up to 25 cm. In embodiments, the lead length is less than 25 centimeters (cm), less than 20 cm, less than 17 cm, less than 15 cm, or less than 10 cm. Further, the lead length is preferably less than or equal to 15 cm. A diameter of the lead can be in a range of 0.1 millimeters (mm) to 5 mm. In one example, a lead of at least 5 cm is inserted into a blood vessel to position the sensor at a distance of 3 to 5 cm within the vessel.

In some embodiments, glucose electronics assembly 304 includes a working electrode 305, a reference electrode 306, and a counter electrode 307. In embodiments, counter electrode 307 has a length greater than the length of working electrode 305. In embodiments, the overall length of each of the working electrode 305, reference electrode 306, and counter electrode 307 ranges from 5 mm to 15 mm. In embodiments, working electrode 305 and reference electrode 306 are preferably spaced from each other at a distance ranging between approximately 4 mm to 8 mm. In some embodiments, reference electrode 306 and counter electrode 307 are positioned outside the epidural space, where the spacing between the working electrode 305 and reference electrode 306 and/or counter electrode 307 is much longer, and may range from 1 mm to 50 mm. In some embodiments, working electrode 305 is at a distance of 1 to 20 mm, preferably at a distance of 3 to 7 mm, and more preferably at 5 mm, from the distal tip of the lead.

A first portion 325 may be further classified into a distal portion comprising glucose electronics assembly 304 and a proximal portion comprising SCS electrode array 302. First portion 325 is in the form of an elongated cylinder, or has a tubular shape, wherein the distal portion has a diameter D1 that is less than a diameter D2 of the proximal portion. In embodiments, a sensing membrane 310 is coated onto the portion of diameter D1 which includes the working electrode 305, the reference electrode 306, and the counter electrode 307. The thickness of the coating of membrane 310 is such that the overall outer diameter of first portion 325 is consistent along its entire length. Moreover, a diameter of first portion 325 is consistent with diameter of extended cylindrical portion within second portion 330 that connects to electronics assembly 315 outside the epidural space.

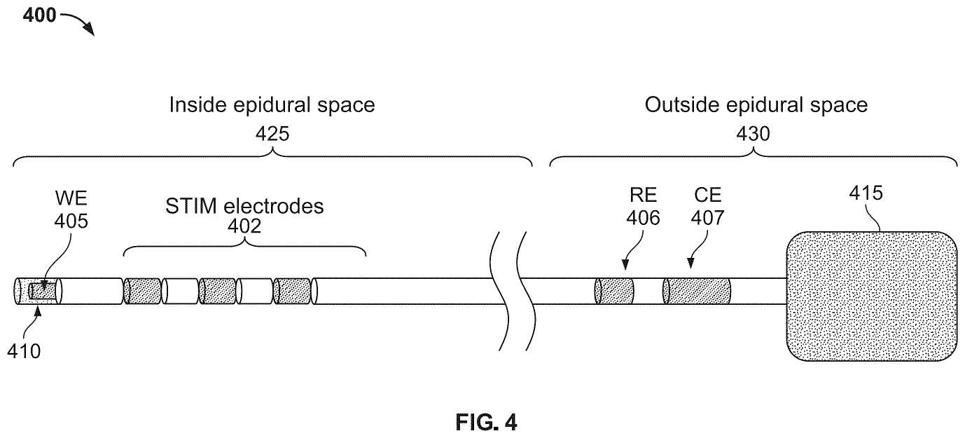

FIG. 4 illustrates another embodiment of a lead configuration 400 including an SCS electrode array 402 for stimulation and a glucose electronic assembly for sensing glucose levels, in accordance with an exemplary embodiment of the present specification. The glucose electronic assembly is formed by a WE 405, an RE 406 and a CE 407. The embodiment illustrated in FIG. 4 is similar to that illustrated in FIG. 3, with the difference in the positions of an RE 406 and a CE 407. A first distal portion 425 that is designed to be inserted within an epidural space comprises a WE 405, and SCS electrode array 402. A second proximal portion 430 that is designed to be positioned outside the epidural space, comprises RE 406, CE 407 and an electronics assembly 415. WE 405, RE 406, and CE 407, together form the glucose electronics assembly. CE 407 has a length greater than a length of WE 405. WE 405 is positioned at the distal-most end of lead configuration 400. A sensing membrane 410 is coated over and above WE 405. A thickness of the coating is selected to make the diameter comprising WE 405 consistent with an outer diameter of the lead body of configuration 400. In embodiments, the lead body comprising electrodes of the glucose electronics assembly and the electrodes of array 402 are in electrical communication with electronics assembly 415. As a result of the positioning of RE 406 and CE 407 on the length of lead configuration 400 so that they are within second proximal portion 430, additional space is available within first distal portion 425 for stimulation electrodes of the array 402 which is also located proximate to WE 405.

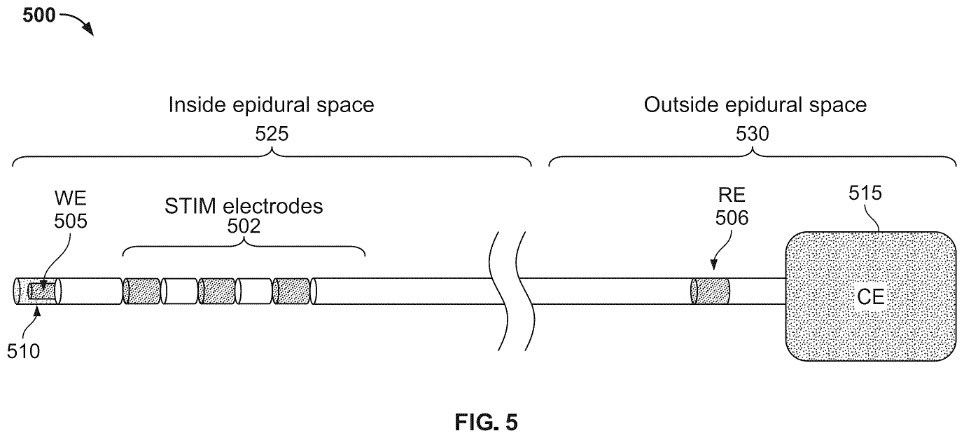

FIG. 5 illustrates yet another embodiment of a lead configuration 500 including a SCS electrode array 502 for stimulation and a glucose electronic assembly for sensing glucose levels, in accordance with an exemplary embodiment of the present specification. The glucose electronic assembly is formed by a WE 505, an RE 506 and a CE 507. The embodiment illustrated in FIG. 5 is similar to that illustrated in FIG. 4, with the difference in the positions of a CE, which is configured within the structure of an electronics assembly 515. A first distal portion 525 that is designed to be inserted within an epidural space comprises a WE 505, and SCS electrode array 502. A second proximal portion 530 that is designed to be positioned outside the epidural space, comprises RE 506 and an electronics assembly 515. Electronics assembly 515 also serves as the CE. It should be noted that the CE is an electrode that balances the function of WE 505. Thus, in embodiments (for electrochemical sensing modalities), WE 505 gains electrons, which are then delivered to body fluids via the CE. WE 505, RE 506, and CE, together form the glucose electronics assembly. WE 505 is positioned at the distal-most end of lead configuration 500. A sensing membrane 510 is coated over and above WE 505. The thickness of the coating is selected to make the diameter comprising WE 505 consistent with an outer diameter of the lead body of configuration 500. In embodiments, the lead body comprising electrodes of the glucose electronics assembly and the electrodes of array 502 are in electrical communication with electronics assembly 515. As a result of the positioning of RE 406 and the CE on the length of lead configuration 500 so that they are within second proximal portion 530, additional space is available within first distal portion 525 for stimulation electrodes of array 502 which is also located proximate to WE 505.

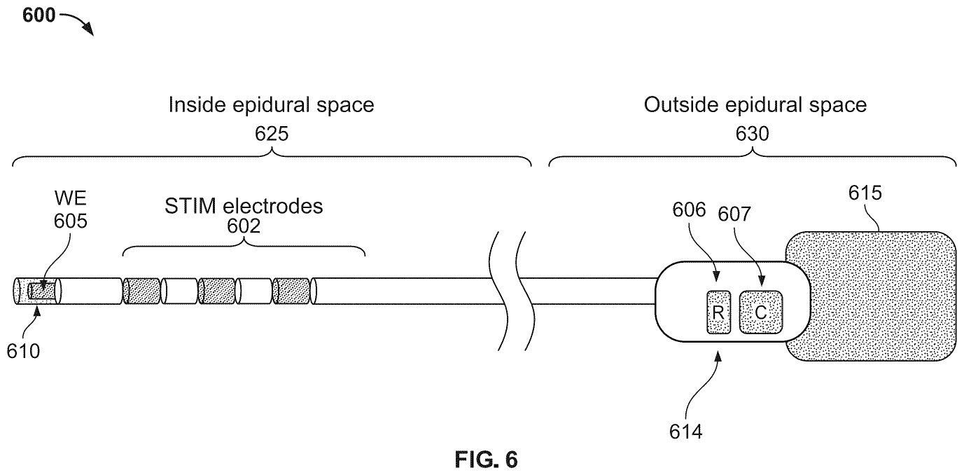

FIG. 6 illustrates another embodiment of a lead configuration 600 including a SCS electrode array 602 for stimulation and a glucose electronic assembly for sensing glucose levels, in accordance with an exemplary embodiment of the present specification. The glucose electronic assembly is formed by a WE 605, an RE 606 and a CE 607. The embodiment illustrated in FIG. 6 is similar to that illustrated in FIG. 4, with the difference in the placement of an RE 606 and a CE 607, which are positioned within an extended structure forming a header 614 of an electronics assembly 615. In some alternative embodiments, RE 606 and CE 607 are positioned on the surface of header 614. A first distal portion 625 that is designed to be inserted within an epidural space comprises a WE 605, and SCS electrode array 602. A second proximal portion 630 that is designed to be positioned outside the epidural space, comprises header 614 including RE 606 and CE 607, where header 614 is connected to electronics assembly 615. Electronics assembly 615 itself is comprised within a housing or a can. Header 614 forms a protruding portion of the housing, protruding in a tubular form towards a distal direction from housing of electronics assembly 615. WE 605 is positioned at the distal-most end of lead configuration 600. A sensing membrane 610 is coated over and above WE 605. The thickness of the coating is selected to make the diameter comprising WE 605 consistent with an outer diameter of the lead body of configuration 600. In embodiments, the lead body comprising electrodes of the glucose electronics assembly and the electrodes of array 602 are in electrical communication with electronics assembly 615. As a result of the positioning of RE 606 and the CE 607 on the length of lead configuration 600 so that they are within second proximal portion 630, additional space is available within first distal portion 625 for stimulation electrodes of array 602 which is also located proximate to WE 605.

While the embodiments illustrated in FIGS. 4 to 6 are exemplary and are designed to provide additional space in the distal first portions of the lead assembly that are meant to be inserted within the epidural space, the positions of RE and CE may differ in other additional embodiments as long as the objective of creating space for the WE and the SCS electrode array is achieved.

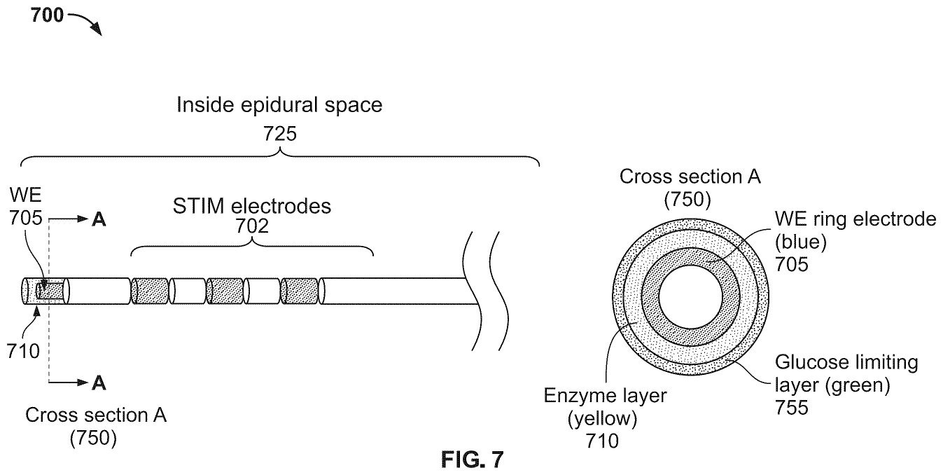

FIG. 7 illustrates a first distal portion (425, 525, 625) 725 of a lead configuration 700, as shown in FIGS. 4, 5, and 6, which is configured to be positioned within an epidural space. The figure further shows a cross-section view 750 of lead configuration 700 at the site of a WE 705. Cross-section 750 is applicable to the site of WE illustrated in FIG. 3 also. The glucose electronics assembly of lead configuration 700 includes WE 705, an RE (not shown), and a CE (not shown). A sensing membrane 710 is coated over and above WE 705. The thickness of the coating is selected to make the diameter comprising WE 705 consistent with an outer diameter of the lead body of configuration 700.

Referring to cross-section view 750, a lumen 760 is surrounded by a ring formed by WE 705. Another ring concentrically shown on an outer surface of WE 705 is sensing membrane 710. In embodiments, the sensing membrane 710 has a thickness ranging from 2 microns to 100 microns, and preferably 40 microns. In some embodiments, another concentric layer of a glucose limiting membrane (GLM) 755 is positioned around an outer surface of sensing membrane 710. GLM 755 can be a polymer-based layer which aids in limiting glucose diffusion and any other interfering species from reaching WE 705. In embodiments, GLM 755 has a thickness ranging from 1 micron to 20 microns, and preferably 10 microns.

### Implantable Pulse Generator (IPG)

SCS systems include either a rechargeable IPG or a non-rechargeable IPG. The two types differ in the type of power source (for example, batteries or newer power sources such as super capacitors or solid state cells) used to operate the IPG. Embodiments of the present specification can be applied to both types of IPGs. In embodiments, a header of the IPG is configured to receive the electrical feedthrough wires and contacts from corresponding sensing electrodes. In some embodiments, a larger feedthrough is designed to accommodate all the wires from the sensing electrodes and the SCS electrodes. The wires of SCS electrodes deliver electrical signals to the destination electrodes, while the wires of the sensing electrodes deliver monitoring signals from the sensing electrodes to the electronics inside the IPG. The electronics in the IPG are configured to accommodate a potentiostat necessary for powering and measuring the electrochemical reaction correlated with glucose concentration, and as communicated through signals from the sensing electrodes.

In some embodiments, the traditional layout of noise-suppressing circuit components in the IPG are modified to avoid current leakage that can distort the glucose sensing signal received from the sensing electrodes. Additionally, firmware implemented by the processing components of the IPG is modified to incorporate instructions to control the potentiostat and measure its signal. The instructions can include those for powering the potentiostat, setting the gain and analog filtering parameters, and acquiring a signal from the sensing electrodes via analog-to-digital conversion. The firmware can additionally incorporate filtering algorithms for the signals from the sensing electrodes. In embodiments, filtering algorithms may include digital filters such as, but not limited to Finite Impulse Response (FIR) and Infinite Impulse Response (IIR). The digital filters are designed and configured to eliminate fundamental and sub-harmonic frequencies caused by the stimulation and/or the associated electronics (which may include the switching network, the power supply, the microprocessor, or other electronic components).

Moreover, the firmware is configured to include instructions to suspend glucose sensor measurement while electrical pulses for spinal-cord stimulation are being delivered. In embodiments, a suspension algorithm is designed or configured to avoid glucose measurement when stimulation related signals are being transmitted as they may add noise to the sensing signal. In some embodiments, the instructions are provided to determine if suspension of glucose measurement is required during stimulation function, and, if so, on which specific (or whether all) stimulation electrodes suspension is required. By way of example, and not to be construed as limiting, if the stimulation pulse train is configured to be cycled on for a duration of 5 msec and off for a duration of 10 msec (repeated cycles), the sensing circuit may be configured to measure during the 10 msec off period. In embodiments, the measurement is taken in the middle of the 10 msec off period to avoid noise from the signal itself but also to avoid any noise just before or after the stimulation signal creation.

The IPG includes components for wireless communication with one or more remote computing devices. The computing devices may include and are not limited to a computing system used by a clinician or a physician, a networking device such as a server, and/or a mobile device such as one used by the patient. The wireless communication components of the IPG are also programmed by the firmware of the IPG. Therefore, the firmware communication scheme with any remote device is programmed to incorporate the glucose sensor parameters, including the current from WE, voltage of CE, WE-RE bias voltage, gain and filter settings, and/or suspension algorithm parameters.

In embodiments, an IPG is configured to incorporate the requirements of a glucose measurement system along with the spinal-cord stimulation system. In some embodiments, two separate, independent electronics systems are employed: a stimulating IPG and a glucose measurement system. The stimulating IPG and glucose measurement system are operated using independent circuits that are configured to be controlled by instructions from independent firmware. The two independent electronic circuits of the IPG and of the glucose measurement system within the SCS are in electronic communication with different electrodes of the proximal portion of the IPG lead interface.

Conventional SCS systems communicate with remote computing devices such as a mobile device through a dedicated application. Mobile apps for patients and physicians are configured to communicate with the IPG. In some cases, a dedicated hardware device communicates with the IPG. In embodiments, the dedicated application, whether on mobile devices or on dedicated hardware devices, are configured to incorporate the data to and from the glucose monitoring device within the SCS. The applications are, in some embodiments, configured to incorporate display features similar to those of a conventional CGM system.

In embodiments, the systems and devices of the present specification are implanted in the same epidural space as the electrodes used in conventional SCS systems. In one embodiment, the conventional procedure to implant a SCS is implemented to implant a combined SCS and glucose measurement system. It should be noted that the addition of a glucose sensing capability does not, in embodiments, require a larger electrode array than an SCS device. However, if the number of sensing electrodes is high (such as for example, greater than three or four), then the resulting lead length caused by the addition of more electrodes is considered during the implantation. In one scenario, if four electrodes are added to an eight-electrode iso-diametric lead, then the implant procedure is modified to account for the necessary additional space in the spinal column for placing a longer lead. This may or may not impact the location of inserting the lead, which translates to the selection of the vertebrae to enter the spinal column. Another consideration is that the epidural space may become narrower farther up the spinal column. In embodiments, for example, the lead insertion location may be positioned at a lower vertebrae to accommodate for a longer lead. In addition, a longer lead delivery tool may be employed.

The above examples are merely illustrative of the many applications of the system of present specification. Although only a few embodiments of the present invention have been described herein, it should be understood that the present invention might be embodied in many other specific forms without departing from the spirit or scope of the invention. Therefore, the present examples and embodiments are to be considered as illustrative and not restrictive, and the invention may be modified within the scope of the appended claims.

What is claimed is:

1. A spinal cord stimulation system, comprising:
a plurality of implantable stimulation electrodes positioned on a substrate;
at least two implantable non-stimulation electrodes configured to continuously monitor at least one physiological parameter, wherein the at least two implantable non-stimulation electrodes are positioned on said substrate;
an implantable pulse generator;
at least one implantable lead electrically connecting the plurality of implantable stimulation electrodes and the at least two implantable non-stimulation electrodes to the implantable pulse generator.

2. The spinal cord stimulation system of claim 1, wherein the at least two implantable non-stimulation electrodes comprise at least a working electrode and at least a reference electrode.

3. The spinal cord stimulation system of claim 2, wherein the at least two implantable non-stimulation electrodes further comprise a counter electrode.

4. The spinal cord stimulation system of claim 2, wherein the reference electrode comprises at least one of a silver substrate or a platinum-iridium substrate.

5. The spinal cord stimulation system of claim 3, wherein a first surface area of the counter electrode is greater than a second surface area of the working electrode.

6. The spinal cord stimulation system of claim 2, wherein the working electrode comprises a polymer-based enzyme coating.

7. The spinal cord stimulation system of claim 6, wherein the working electrode further comprises a polymer-based coating over the polymer-based enzyme coating.

8. The spinal cord stimulation system of claim 1, wherein the system comprises two sets of the at least two implantable non-stimulation electrodes, wherein the two sets are configured for differential measurement of the at least one physiological parameter.

9. The spinal cord stimulation system of claim 1, wherein the at least one physiological parameter comprises glucose levels.

10. The spinal cord stimulation system of claim 1, wherein the at least two implantable non-stimulation electrodes are configured to continuously monitor the least one physiological parameter within an epidural space.

11. The spinal cord stimulation system of claim 1, wherein the implantable pulse generator comprises a lead assembly, wherein the lead assembly comprises electrical contacts for electrical communication with the plurality of implantable stimulation electrodes and with the at least two implantable non-stimulation electrodes.

12. The spinal cord stimulation system of claim 1, wherein the implantable pulse generator comprises at least one of a potentiostat, an analog to digital converter, a power source, a digital communication circuit, or a microcontroller.

13. The spinal cord stimulation system of claim 1, wherein the implantable pulse generator comprises a power source that is at least one of rechargeable or non-rechargeable.

14. The spinal cord stimulation system of claim 1, wherein the implantable pulse generator comprises a programmable electronic circuitry configured to communicate with at least one computing device.

15. The spinal cord stimulation system of claim 1, wherein the at least two implantable non-stimulation electrodes form a sensor for continuously monitoring glucose levels in a patient, wherein the sensor is at least one of an enzymatic sensor or a non-enzymatic sensor.

16. The spinal cord stimulation system of claim 15, wherein the enzymatic sensor comprises at least one of a wired enzymatic sensor, an engineered enzymatic sensor, an H2O2 based enzymatic sensor, or an O2 differential based enzymatic sensor.

17. The spinal cord stimulation system of claim 15, wherein the non-enzymatic sensor comprises at least one of a photodetector or a glucose binding molecule.

18. The spinal cord stimulation system of claim 1, wherein the at least two implantable non-stimulation electrodes comprise one or more bioresorbable membranes.

19. A continuous physiological parameter monitoring device integrated with a spinal cord stimulation system, comprising:
a plurality of implantable stimulation electrodes positioned on a substrate;
at least two implantable non-stimulation electrodes configured to continuously monitor at glucose levels positioned on said substrate;
an implantable pulse generator;
at least one implantable lead electrically connecting the plurality of implantable stimulation electrodes and the at least two implantable non-stimulation electrodes to the implantable pulse generator.

20. A spinal cord stimulation system, comprising:
a plurality of implantable stimulation electrodes positioned on a substrate;
at least one implantable non-stimulation electrode configured to continuously monitor at least one physiological parameter, wherein the at least one implantable non-stimulation electrodes are positioned on said substrate;
an implantable pulse generator;
at least one implantable lead electrically connecting the plurality of implantable stimulation electrodes and the at least one implantable non-stimulation electrode to the implantable pulse generator.

21. The spinal cord stimulation system of claim 20, wherein the at least one implantable non-stimulation electrodes comprises a working electrode.

22. The spinal cord stimulation system of claim 20, further comprising at least two non-stimulation electrodes that are not implanted.

23. The spinal cord stimulation system of claim 22, wherein the at least two non-stimulation electrodes that are not implanted further comprise a reference electrode and a counter electrode.
